(19) United States
(12) Patent Application Publication
(10) Pub. No.: US 2004/0101436 A1
(43) Pub. Date: May 27, 2004

(54) Methods for sterilizing biological materials

(75) Inventors: Martin J. MacPhee, Montgomery Village, MD (US); Randall S. Kent, Thousand Oaks, CA (US); Edward A. Horton, Toronto (CA); Dawson Beall, Gaithersburg, MD (US)

Correspondence Address: FLESHNER & KIM, LLP, P.O. BOX 221200, CHANTILLY, VA, 20153, US

(73) Assignee: Clearant, Inc.

(21) Appl. No.: 10/694,733

(22) Filed: Oct. 29, 2003

Related U.S. Application Data
(63) Continuation of application No. 09/533,547, filed on Mar. 23, 2000.
Application No. 09/533,547 is a continuation-in-part of application No. 08/573,149, filed on Dec. 15, 1995, now Pat. No. 6,171,549.
Application No. 08/573,149 is a continuation-in-part of application No. PCT/CA94/00401, filed on Jul. 22, 1994.
Application No. PCT/CA94/00401 is a continuation-in-part of application No. 08/095,698, filed on Jul. 22, 1993, now Pat. No. 5,362,442.

(51) Int. Cl. A61L 2/08; A01N 1/02

(52) U.S. Cl. 422/22; 435/2

(57) ABSTRACT

Methods are disclosed for sterilizing biological products to reduce the level of active biological contaminants such as viruses, bacteria, yeasts, molds, mycoplasmas and parasites.

This application is a continuation-in-part of prior U.S. patent application Ser. No. 08/573,149, the disclosure of which is herein incorporated by reference in its entirety.

### FIELD OF THE INVENTION

The present invention relates to methods for sterilizing biological materials to reduce the level of active biological contaminants therein, such as viruses, bacteria, yeasts, molds, mycoplasmas and/or parasites.

### BACKGROUND OF THE INVENTION

Several products that are prepared from human, veterinary or experimental use may contain unwanted and potentially dangerous contaminants such as viruses, bacteria, yeasts, molds, mycoplasmas and parasites. Consequently, it is or utmost importance that any biologically active contaminant in the product be inactivated before the produce is used. This is especially critical when the product is to be administered directly to a patient, for, example in blood transfusions, organ transplants and other forms of human therapies. This is also critical for various biotechnology products which are grown in media which contain various types of plasma and which may be subject to mycoplasma or other viral contaminants.

Previously, most procedures have involved methods that screen or test products for a particular contaminant, rather than removal or inactivation of the contaminant from the product. Products that test positive for a contaminant are merely not used. Examples of screening procedures include the testing for a particular virus in human blood from blood donors. Such procedures, however, are rot always reliable and are not able to detect the presence of viruses in very low numbers. This reduces the value or certainty of the test in view of the consequences associated with a false negative result. False negative results can be life threatening in certain cases, for example in the case of Acquired Immune Deficiency Syndrome (AIDS). Furthermore, in some instances it can take weeks, if not months, to determine whether or not the product is contaminated.

More recent efforts have focused in methods to remove or inactivate contaminants in the products. Such methods include heat treating, filtration and the addition of chemical inactivants or sensitizers to the product. Feat treatment requires that the product be heated to approximately 60° C. for about 70 hours which can be damaging to sensitive products. Heat inactivation can destroy up to 50% of the biological activity of the product. Filtration involves filtering the product in order to physically remove contaminants. Unfortunately this method may also remove products that have a high molecular weight. Farther, in certain cases small viruses may not be removed by the filter because of the large molecular structure of the product. The procedure of chemical sensitization involves the addition of noxious agents which bind to the DNA/RNA of the virus and which are activated either by UV or ionizing radiation to produce free radicals which break the chemical bonds in the backbone of the DNA/RNA of the virus or complex it in such a way that the virus can no longer replicate This procedure requires that unbound sensitizer is washed from cellular products since the sensitizers are toxic, if not mutagenic or carcinogenic, and can not be administered to a patient.

Irradiating a product with gamma radiation is another method of sterilizing a product. Gamma radiation is effective in destroying viruses and bacteria when given in high total, doses (Keathly et al., “Is There Life After Irradiation? Part 2,” BioPharm July-August, 1993, and Leitman, USe of Blood Cell Irradiation in the Prevention of Post Transfusion Graft-vs-Host Disease,” Transfusion Science 10:219-239 (1989)). The published literature in this area, however, teaches that gamma radiation can be damaging to radiation sensitive products, such as blood. In particular, it has been shown that high radiation doses are injurious to red cells, platlets and granulocytes (Leitman). U.S. Pat. No. 4,620,908 discloses that protein products must be Frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective.” Unfortunately, manly sensitive biologicals, such as blood, would lose viability and activity if subjected to freezing for irradiation purposes and then thawing prior to administration to a patient.

In view of the difficulties discussed above, there remains a need for methods of sterilizing biological materials that are effective for reducing the level of active biological contaminants without an adverse effect on the biological material.

### SUMMARY OF THE INVENTION

Accordingly, it is an object of the present invention to provide methods of sterilizing biological materials by reducing the level of active biological contaminants without adversely effecting the biological material. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for sterilizing a biological material that is sensitive to ionizing radiation comprising: (i) reducing the residual solvent content of a biological material to a level effective to protect the biological material front ionizing radiation; and (ii) irradiating the biological material with radiation at an effective rate for a time effective to sterilize the biological material.

A second embodiment of the present invention is directed to a method for sterilizing a biological material that is sensitive to ionizing radiation comprising: (i) adding to a biological material at least one stabilizer in an amount effective to protect the biological material from ionizing radiation; and (ii) irradiating the biological material with radiation at an effective rate for a time effective to sterilize the biological material.

A third embodiment of the present invention is directed to a method for sterilizing a biological material that is sensitive to ionizing radiation comprising: (i) reducing the residual solvent content of a biological material to a level effective to protect, the biological material from ionizing radiation: (ii) adding to the biological material at least one stabilizer in an amount effective to protect the biological material from ionizing radiation; and (iii) irradiating the biological material with radiation at an effective rate for a time effective to sterilize the biological material.

### DETAILED DESCRIPTION OF THE PREFERRED EMBODIMENTS

### A. Definitions

Unless defined otherwise, all technical and scientific tens used herein are intended to have the same meaning as is commonly understood by one of ordinary skill in the relevant art. All patents and publications mentioned herein are expressly incorporated by reference.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins: botanicals; foods and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs for transplant, such as hearts, lungs, liver, kidney, intestine, pancreas, limbs and digits; lipids; carbohydrates; collagen (native, afibrillar, atelomeric, soluble and insoluble); chitin and its derivatives including chitosan and its derivatives including NO-carboxy chitosan (NOCC); stem cells, islet of langerhans cells, and other cellular transplants, including genetically altered cells; red blood cells; white blood cells, including monocytes and stem cells; and platelets.

As used herein, the term “sterilize” is intended to mean a reduction in the level of at least one active biological contaminant found in the biological material being treated according to the present invention.

As used herein, the term “biological contaminant” is intended to mean a contaminant that, upon direct or indirect contact with a biological material, may have a deleterious effect on a biological material. Such biological contaminants include the various viruses, bacteria and parasites known to those of skill in the art to generally be found in or infect biological materials such as whole blood or blood components. Examples of biological contaminants include, but are not limited to, the following: viruses, such as human immunodeficiency viruses and other retroviruses, herpes viruses, paramyxoviruses, cytomegaloviruses, hepatitis viruses (including hepatitis B and hepatitis C), pox viruses, toga viruses, Ebstein-Barr virus and parvoviruses: bacteria, such as Escherichia, Bacillus, Campylobacter, Streptococcus and Staphalococcus; parasites, such as Trypanosoma and Malarial parasites, including Plasmodium species; yeasts; molds; mycoplasmas; and prions. As used herein, the term “active biological contaminant” is intended to mean a biological contaminant that is capable of causing the deleterious effect.

As used herein, the term “blood components” is intended to mean one or more of the components that may be separated From whole blood and include, but are not limited to, cellular blood components, such as red blood cells, white blood cells and platelets; blood proteins, such as blood clotting factors, enzymes, albumin, plasminogen, fibrinogen and immunoglobulins; and liquid blood components, such as plasma and plasma-containing compositions.

As used herein, the term “cellular blood component” is intended to mean one or more of the components of whole blood that comprises cells, such as red blood cells, white blood cells or platelets.

As used herein, the tern “blood protein” is intended to mean one or more of the proteins that are normally found in whole blood. Illustrative examples of blood proteins found in mammals (including humans) include, but are not limited to, coagulation proteins (both vitamin K-dependent, such as Factor VII or Factor IX, and non-vitamin K-dependent, such as Factor VIII and von Willebrands factor), albumin, lipoproteins (high density lipoproteins and/or low density lipoproteins), complement proteins, globulins (such as immunoglobulins IgA, IgM, IgG and IgE), and the like. A preferred group of blood proteins include Factor I (Fibrinogen), Factor II (Prothrombin), Factor III (Tissue Factor), Factor IV (Calcium) Factor V (Proaccelerin), Factor VI (Accelarin), Factor VII (Proconvertin, serum prothrombin conversion), Factor VIII (Antihemophiliac factor A), Factor IX (Antihemophiliac factor B), Factor X (Stuart-Prower Factor), Factor XI (Plasma thromboplastin antecedent), Factor XII (Hageman Factor), Factor XIII (Protansglutamidase), von Willebrand Factor (vWF), Factor Ia, Factor IIa, Factor Va, Factor VIa, Factor VIIa, Factor VIIa, Factor IXa, Factor Xa, and Factor XIIIa.

As used herein, the term “liquid blood component” is intended to mean one or more of the fluid, non-cellular components of whole blood, such as plasma (the fluid, non-cellular portion of the blood of humans or animals as found prior to coagulation) or serum (the fluid, non-cellular portion of the blood of humans or animals after coagulation).

As used herein, the term “a biologically compatible solution” is intended to mean a solution to which biological materials may be exposed, such as by being suspended or dissolved therein, and remain viable, i.e., retain their essential biological and physiological characteristics. Such biologically compatible solutions preferably contain an effective amount of at least one anticoagulant.

As used herein, the term “a biologically compatible buffered solution” is intended to mean a biologically compatible solution having a pH and osmotic properties (e.g, tonicity, osmolality and/or oncotic pressure) suitable for maintaining the integrity of biological materials. Suitable biologically compatible buffered solutions typically have a pH between 5 and 8.5 and are isotonic or only moderately hypotonic or hypertonic. Biologically compatible buffered solutions are known and readily available to those of skill in the art.

As used herein, the term “stabilizer” is intended to mean a compound or material that reduces any damage to the biological material being irradiated to a level that is insufficient to preclude the safe and effective use of that material. Illustrative examples of stabilizers include, but are not limited to, the following: antioxidants, such as ascorbic aced and tocopherol; and free radical scavengers, such as ethanol, including Type I and Type II free radical scavengers, preferably at least one Type I and at least one Type II free radical scavenger. Preferred examples of stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate (DL-6,8-dithioloctanoic acid methyl ester), lipoamide, bisonor methyl ester and tatranor-dihydrolipoic acid, furan fatty acids, oleic and linoleic and palmitic acids and their salts and derivatives; flavonoids, phenylpropaniods, and flavenols, such as quercetin, rutin and its derivatives, apigenin, amioflavone, catechin, hesperidin and, naringin, carotenes, including beta-carotene; Co-Q010; xanthophylls; polyhydric alcohols, such as glycerol, mannitol; sugars, such as xylose, glucose, ribose, mannose, fructose and trehalose, amino acids, such as histidine, N-acetylcysteine (NAC), glutamic acid, tryptophan, sodium capryl N-acetyl tryptophan and methionine; azides, such as sodium azide; enzymes, such as Superoxide Dismutase (SOD) and Catalase; uric acid and its derivatives, such as 1,3-dimethyluric acid and dimethylthiourea; allopurinol; thiols, such as glutathione and cysteine; trace elements, such as selenium; vitamins, such as vitamin A, vitamin C (including its derivatives and salts such as sodium ascorbate and palmitoyl ascorbic acid) and vitamin E (and its derivatives and salts such as tocopherol acetate and alpha-tocotrienol); chromanol-alpha-C6; 6-hydroxy-2,5,7,8-tetramethylchroma-2 carboxylic acid (Trolox) and derivatives; extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS), 1,2-dithiane-4,5-diol; reducing substances, such as butylated hydroxyanisole (BHA) and butylated hydroxytoluene (BHT); cholesterol; probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); and 4-nydroxy-2,2, 6,6-Tetramethylpiperidin-1-oxyl (Tempol).

As used herein, the term “residual solvent content” is intended to mean the amount of freely-available liquid in the biological material. Freely-available liquid means that liquid, such as water or an organic solvent (e.g. ethanol, isopropanol, polyethylene glycol, etc.), present in the biological material that is not bound to or complexed with one or more of the non-liquid components of the biological material (e.g. proteins, metal ions or salts, etc.). Freely-available liquid includes intracellular water. The residual solvent contents referenced herein refer to levels determined by the FDA approved, modified Karl Fischer method (Meyer and Boyd, Analytical Chem., 31, 215-219, 1959; May, et al., J. Biol. Standardization, 10, 249-259, 1982; Centers for Biologics Evaluation and Research, FDA, Docket No. 89D-0140, 83-93; 1990).

As used herein, the term “sensitizer” is intended to mean a substance that selectively targets viral, bacterial, and/or parasitic contaminants, rendering them more sensitive to inactivation by radiation, therefore permitting the use of a lower rate of radiation and/or a shorter time of irradiation than in the absence of the sensitizer. Illustrative examples of suitable sensitizers include, but are not limited to, the following; psoralen arid its derivatives and analogs (including 3-carboethoxy psoralens), angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphorins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide.

As used herein, the term “proteinaceous material” is intended to mean a cellular material that comprises at least one protein or peptide. This material is preferably composed primarily of protein(s) and/or peptide (s). It may be a naturally occurring material, either in its native state or following processing/purification and/or devatization. It may be artificially produced, either by chemical synthesis or utilizing recombinant/transgenic technology. Such artificially produced material may also be processed/purified and/or derivatized. Illustrative examples of proteinaceous materials include, but are not limited to, the following: proteins/peptides produced from tissue culture; milk (dairy products); ascites; hormones; growth factors; materials, including pharmaceuticals, extracted or isolated from animal tissue (such as heparin and insulin) or plant matter; plasma (including Fresh, frozen and freeze-dried); fibrinogen, fibrinogen derivatives, fibrin, fibrin I, fibrin II, soluble fibrin and fibrin monomer, and/or fibrin sealant products; whole blood; protein C; protein S; alpha-1 anti-trypsin (alpha-1 protease inhibitor): butyl-cholinesterase; anticoagulants, such as coumarin drugs (warfarin): streptokinase; tissue plasminogen activator (TPA); erythropoietin (EPO); urokinase; neupogen; anti-thrombin-3: alpha-glucosidase; (Fetal) Bovine Serum/Horse Serum; meat; immunoglobulins, including anti-sera, monoclonal antibodies, polyclonal antibodies and genetically engineered or produced antibodies; albumin; alpha-globulins; beta-globulins; gamma-globulins; coagulation proteins; complement proteins; and interferons.

As used herein, the term “ionizing radiation” is intended to mean radiation of sufficient energy to ionize (produce ions) the irradiated biological material. Types of ionizing radiation include, but are not limited to, the following: (i) corpuscular (streams of subatomic particles such as neutrons, electrons (including e-beam radiation), and/or protons): and (ii) electromagnetic (originating in a varying electromagnetic field, such as radio waves, visible and invisible light (including ultraviolet), x-radiation, and gamma rays).

### B. Particularly Preferred Embodiments

A first preferred embodiment of the present invention is directed to a method for sterilizing a biological material that is sensitive to ionizing radiation comprising: (i) reducing the residual solvent content of a biological material to a level effective to protect the biological material from ionizing radiation;: and (ii) irradiating the biological material with radiation at an effective rate for a time effective to sterilize the biological material.

A second embodiment of the present invention is directed to a method for sterilizing a biological material that is sensitive to ionizing radiation comprising:

(i) adding to a biological material at least one stabilizer in an amount effective to protect the biological material From ionizing radiation; and (ii) irradiating the biological material with radiation at an effective rate for a time effective to sterilize the biological material.

A third embodiment of the present invention is directed to a method for sterilizing a biological material that is sensitive to ionizing radiation comprising: (i) reducing the residual solvent content of a biological material to a level effective to protect the biological material from ionizing radiation; (ii) adding to the biological material at least one stabilizer in an amount effective to protect the biological material from ionizing radiation: and (iii) irradiating the biological material with radiation at an effective rate For a time effective to sterilize the biological material. The order of steps (i) and (ii) may, of course, be reversed as desired.

The biological material sterilized in accordance with the methods of the present invention may be any material obtained or derived from a living or deceased organism, including a solid material or liquid material or a suspension of any solid(s) in any liquid(s) or a coating of any solid or liquid on a biological or non-biological substrate.

According to the methods of the present invention, the residual solvent content of the biological material is reduced prior to irradiation of the biological material with ionizing radiation. The residual solvent content is reduced to a level that is effective to protect the biological material from the ionizing radiation. Suitable levels of residual solvent content may vary depending upon the nature and characteristics of the particular biological material being irradiated and can be determined empirically by one skilled in the art. Preferably, when the solvent is water, the residual solvent content is less than about 2.0%, more preferably less than about 1.0%, even more preferably less than about 0.5% and most preferably less than about 0.2%.

While not wishing to be bound by any theory of operability, it is believed that the reduction in residual solvent content reduce the degrees of freedom of the biological material and thereby protects it from the effects of the ionizing radiation. Similar results might therefore be achieved by lowering the temperature of the biological material below its eutectic point or below its freezing point to likewise reduce the degrees of freedom of the biological material. These results permit the use of a higher rate of irradiation than might otherwise be acceptable.

The residual solvent content of the biological material may be reduced by any of the methods and techniques known to those skilled in the at for removing solvent from a Biological material. A particularly preferred method for reducing the residual: solvent content of a biological material is lyophilization. According to a particularly preferred embodiment of the present invention, a biological material which has been lyophilized is stored under vacuum or an inert atmosphere (preferably a noble gas, such as helium or argon, more preferably a higher molecular weight noble gas, and most preferably argon) prior to irradiation.

The ionizing radiation employed in the present invention may be any ionizing radiation effective for the inactivation of one or more biological contaminants of the biological material being treated. Preferably the ionizing radiation is electromagnetic radiation and a particularly preferred form of ionizing radiation is gamma radiation.

According to the methods of the present invention, the biological material is irradiated with the ionizing radiation at a rate effective for the inactivation of one or more biological contaminants of the biological material. Suitable rates of irradiation may vary depending upon the particular form of ionizing radiation and the nature and characteristics of the particular biological material being irradiated and the particular biological contaminants being inactivated. Suitable rates of irradiation can be determined empirically by one skilled in the art. Preferably, the rate of irradiation is constant For the duration of the sterilization procedure.

According to a particularly preferred embodiment of the present invention, the rate of irradiation is not more than about 3.0 key/hour, more preferably between about 0.1 kGy/hr. and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particularly preferred embodiment oil the present invention, the rate of irradiation is at least about 3.0 kGy/hr., more preferably at least about 6 kGy/hr., even more preferably at least about 16 kGy/hr. and most preferably at least about 30 kGy/hr.

The biological material is irradiated with the ionizing radiation for a time effective for the inactivation of one or more biological contaminants of the biological material Suitable irradiation times may vary depending upon the particular form and rate of ionizing radiation and the nature and characteristics of the particular biological material being irradiated and the particular biological contaminants being inactivated. Suitable irradiation times can be determined empirically by one skilled in the art.

Optionally, an effective amount of at least one sensitizer is added to the biological material prior to irradiation with ionizing radiation. Suitable sensitizers are known to those skilled in the art.

According to methods of the present invention, the irradiation of the biological material may occur at any temperature which is not deleterious to the biological material being treated. According to a preferred embodiment, the biological material is irradiated at ambient temperature. According to an alternate preferred embodiment, the biological material is irradiated at reduced temperature, preferably at or below the eutectic point of the biological material.

### C. EXAMPLES

The following examples are illustrative, but not limiting, of the present invention. Other suitable modifications and adaptations are of the variety normally encountered by those skilled in the art and are fully within the spirit and scope of the present invention.

### Example b 1

### Sterilization of Blood

A 200 ml bag of one day old packed red blood cells was used. Ethanol was added to the cells in order to achieve a final ethanol concentration of 0.01% v/v. The red blood cells were diluted by a factor of one in ten using a modified Citrate Phosphate Dextrose (CPD) solution having a pH of about 6.4 to 6.7 and having the following composition in a total volume of 500 ml:

| | |
|---|---|
| Citrate Acid Monohydrate | 0.2 g |
| Sodium Citrate Dihydrate | 27.3 g |
| Sodium Monobasic | 2.2 g |
| Phosphate | |
| Sodium Dibasic Phosphate | 1.0 g |
| Dextrose | 3.2 g |

The cells were irradiated in a commercial size gamma irradiator which contained a cobalt 60 source rack. Irradiation was done off carrier in an unprotected box. The cells were irradiated for twenty-four hours at a rate of approximately 1: kGy/hr. After the irradiation period the red blood cells were examined visually and were found to be viable, having a brilliant red color. A control sample, consisting of packed red blood cells that were not diluted with the above-described CPD solution, was not viable after irradiation.

Four days after the irradiation procedure, the diluted cells were tested for levels of various blood components and the results are shown in Table 1. The control sample consisted of blood from the same bag as the test sample but it did not undergo irradiation. Table 1 illustrates that dilution and irradiation of human blood cells did not significantly alter the white blood cell count. The platelet count and hematocrit values were slightly lower than the control; however, these values are still within the range that is seen in normal adult blood. The level of hemoglobin was higher than in the control indicating that some red blood cells did lyse during the procedure. This is also evidenced by lower red blood cell count. Nevertheless, contrary to what has been previously published, up to 50 kGy of radiation did not destroy the components of blood by the present procedure. The cells were also counted and found to be viable after 25 kGy of gamma irradiation delivered at a low dose rate of 1 kGy/hr.

TABLE 1

| Component | Irradiated Blood | Control Blood |
|---|---|---|
| White Blood Cells | 4 K/mm³ | 4.8 K/mm³ |
| Red Blood Cells | 3 Mi/mm³ | 7.2 Mi/mm³ |
| Hemoglobin | 42 g/dl | 21 g/dl |
| Hematocrit | 46% | 64% |
| Platelet | 100 k/mm³ | 120 k/mm³ |

### Example 2

### Sterilization of Dextrose

Dextrose (or glucose) containing solutions are used in the treatment of carbohydrate and fluid depletion, in the treatment of hypoglycemia, as a plasma expander, in renal dialysis and to counteract hepatotoxins (The Merck Index, Eleventh Edition, Merck & Co., Inc. (1989), and Martindale's Extra Pharmacopecia, p.1, 265). Dextrose is also the preferred source of carbohydrate in parental nutrition regiments (The Merck Index, Eleventh Edition, Merck & Co., Inc. (1989), and Martindale's Extra Pharmacopecia, p.1, 265). In all of the above applications, the dextrose must be sterilized before use. Sterilization of dextrose-containing products is generally done by heat sterilization or autoclaving. Unfortunately, these methods have been reported to degrade or carmelize dextrose-containing solutions resulting in a color change in the solution (Martindale's Extra Pharmacopecia p.1, 265). Gamma irradiation of glucose has also been reported to decompose glucose-containing solutions (Kawakishi, et al., “Radiation-Induced Degradation of D-glucose in Anaerobic Contition,” Agric. Biol. Chem., June 1977). Therefore, there is a need for a method that can sterilize dextrose-containing products that does not degrade the product itself. In view of the problems of the prior art, a dextrose solution was treated according to the method of the present invention as follows.

A 5% dextrose solution was irradiated for 24 hours, at a rate of approximately 1 kGy/hr. After irradiation the product was tested and it was found that there was no visible light spectrum change as compared to the non-irradiated control. Therefore, the present method can be useful in sterilizing products that contain dextrose.

In addition to the above experiment, fresh solutions of 5% and 50% dextrose were irradiated to 25 kGy over 36 hours at ambient temperature. The results were similar to those described above. In addition, UV/VIS scans were obtained and demonstrated a complete absence of the peak at 283.4 nm for “furfural” as per U.S.P. In contrast, dextrose samples sterilized using an autoclave contain the 283.4 furfural peak. “Furfurals” are carcinogenic.

### Example 3

### Sterilization of Human Serum Albumin

Normal Human Serum Albumin was irradiated as a 25% salt-poor solution to a total dose of 25 kGv over 36 hours using a Gamma cell 220 (Co⁶⁰ is the gamma ray source in this instrument). The temperature was not controlled during the irradiation but it is estimated that the container holding he albumin solution was approximately 23° C. The results of HPLC analysis are given in Table 2.

TABLE 2

| Parameter | Control (%) | Irradiated (%) |
|---|---|---|
| Polymer | 2 | 3 |
| Dimer | 7 | 8 |
| Monomer | 90 | 86 |
| Low Molecular | 1 | 3 |
| Weight | | |
| pH | 7.05 | 6.97 |
| NTU (must be > 20) | 11.4 | 11.4 |

As the results demonstrate, Normal Human Serum Albumin can safely be irradiated to 25 kGy (at a rate of approximately 0.7 kGy/hr) at room temperature without adversely affecting the essential properties of the protein. This has not been demonstrated before. All other attempts at irradiating serum albumin require that it be irradiated in the frozen stage. This adds to the cost and difficulty of doing the irradiation.

### Example 4

Normal human blood from a healthy donor was taken in a heparinized tube, washed three times with standard CPD solution, then diluted 1:20 with COD containing 0.01% v/v Ethanol. This latter solution of CPD with 0.01% v/v Ethanol is called SCPD. Two ml aliquots were then placed in 10 ml plastic test tubes and irradiated to different doses up to 26 kGy over 36 hours at room temperature. There was no haemolysis and the cells appeared intact if somewhat large and slightly irregular in shape. The results of three separate experiments are reported in Table 3.

TABLE 3

| Parameter | RCB¹ | HGB² | HCT³ | MCV⁴ | MCH⁵ | MCHC⁶ | RDW⁷ | Flags |
|---|---|---|---|---|---|---|---|---|
| 1* | 1.08 | 41 | .097 | 89.5 | 38.3 | 427 | 17.7 | Nearly |
| Control | .99 | 33 | 0.89 | 90.2 | 33.0 | 366 | 15.3 | Normal |
| 2* | | | | 95.0 | 32.3 | 339 | 12.0 | |
| 12 kGy 1 | 1.22 | 45 | .166 | 135.8 | 36.5 | 269 | 27.3 | 1 + Anisocytosis |
| | 1.38 | 45 | .199 | 144.7 | 33.0 | 228 | 24.9 | 3 + Macrocytocis |
| 1 | 1.04 | 32 | .169 | 163.0 | 31.3 | 152 | 18.8 | 1 + Anisocytosis |
| 16 kGy | 0.54 | 29 | .088 | 162.5 | 54.5 | 335 | 18.8 | 3 + Macrocytocis |
| 2 | 0.82 | 27 | .128 | 156.5 | 32.8 | 209 | 19.8 | 2 + Anisocytosis |
| | 0.81 | 26 | .124 | 152.6 | 32.4 | 212 | 20.2 | 3 + Macrocytocis |
| 1 | 0.79 | 244 | .125 | 158.4 | 30.8 | 194 | 19.4 | 1 + Anisocytosis |
| 20 kGy | 1.26 | 28 | .203 | 161.5 | 22.1 | 137 | 19.0 | 3 + Macrocytocis |
| 2 | 0.93 | 30 | .141 | 151.5 | 32.3 | 213 | 20.1 | 2 + Anisocytosis |
| | 0.92 | 30 | .143 | 155.5 | 32.1 | 207 | 20.5 | 3 + Macrocytocis |
| 26 kGy 1 | 1.15 | 34 | .180 | 155.9 | 29.4 | 189 | 19.1 | 1 + Anisocytosis |
| | 1.15 | 34 | .176 | 153.0 | 29.9 | 195 | 23.4 | 3 + Macrocytocis |

*Experiment 1 and Experiment 2
¹Red Blood Cell Count: Cells x10¹²/liter
²Hemoglobin: grams/liter
³Hematocrit
⁴Mean Corpuscular Volume: Femtoliters
⁵Mean Corpuscular Hemoglobin: picograms
⁶Mean Corpuscular Hemoglobin Concentration: grams/liter

The cells were easily put into suspension and reconstituted in fresh buffer.

The following three experiments (Examples 5, 6 and 7) were conducted in order to determine the efficacy of the method when treating HIV-contaminated blood. In each Example the cells were similarly treated. In these experiments, the cells were gently agitated after 12, 16 and 24 hours of irradiation. Further, in the third experiment (Example 7), the cells were placed in T25 flasks to provide greater surface area and reduce the concentration due to settling in the bottom of the centrifuge tubes. In each case, the cells were irradiated at a dose rate of approximately 0.7 kGy/hr.

### Example 5

### Sterilization of HIV-Containing Blood

The following experiments were undertaken with the following specific objectives:

1. To evaluate the toxicity of the process towards red blood cells (RBCs).

2. To evaluate the anti-retroviral activity of the process.

### Procedure

Initially, 2 ml of anticoagulated blood was obtained from an HIV-seronegative donor. The blood was centrifuged, and the plasma was removed. The remain ng cell pellet was resuspended in 10 ml of the CPD buffer and centrifuged. This washing process was repeated a total of three times. The final pellet was resuspended in 40 ml of the SCPD buffer, and distributed into plastic tubes in 2 ml aliquots, with 16 separate aliquots being retained for further manipulation. For 8 of these tubes, an aliquote of HTLV-III3 was added. This is a laboratory strain of the HIV virus and 100 tissue culture infective doses (TCID) were added to each of the tubes to be infected. For the remaining 8 tubes, a “mock” infection was performed, by adding a small amount of non-infectious laboratory buffer, phosphate buffered saline (PBS). Four infected and four non-infected tubes were subjected to the process. For comparison, the remaining 8 tubes (four infected and four non-infected) were handled in an identical manner, except that they were not subjected to the process.

It should be stated that at the beginning of the study, a separate aliquot of blood was obtained from the donor. This was processed in the clinical hematology laboratory aid a complete hemogram was performed. These baseline results were compared to repeat testing on the study aliquots, which included evaluation of four processed and four unprocessed samples, all of which were not infected with HIV.

An aliquot of 0.5 ml of each of the infected study samples was inoculated on mononuclear cells (MCs) which had been obtained three days earlier. These cells had been suspended in RMPI culture medium, with 10% fetal calf serum and other additives (penicillin, streptomycin, glutamine and HEPES buffer) along with 1 μg/ml PHA-P. At the same time as this inocculation, the cells were resuspended in fresh medium with rIL-2 (20 U/ml). The cultures were maintained for 7 days. Twice weekly, a portion of the culture medium was harvested for the measurement of HIV p24 antigen levels (commercial ELISA kit, Coulter Electronics, Hialeah, Fla.) for the measurement or viral growth.

A separate aliquot of the eight infected study samples was used for viral titration experiments. Briefly, serial four-fold dilutions of the virus-containing fluids (ranging from 1:16 to 1:65,536) were inoculated in triplicate in 96-well flat-bottom tissue culture plates. PHA-stimulated MCs were added to each well (4 million cells in 2 ml culture medium, with IL-2). An aliquot of the supernatant from each culture well was harvested twice weekly for the measurement of HIV p24 antigen levels. A well was scored as “positive” if the HIV p24 antigen value was >30 pg/ml.

The viral titer was calculated according to the Spearman-Karber method (se ACTG virology protocol manual) using the following equation:

M=xk+d[0.5−(1/n)r]

M: titer (in log 4)

xk: dose of highest dilution

d: space between dilutions

n: number of wells per dilution

r: sum of total number of wells.

### Results

Red blood cell parameters for the baseline sample as well as for the unprocessed and processed study samples are shown Table 4,

TABLE 4

| Sample/Number | MCV | MCH | MCHC |
|---|---|---|---|
| Baseline | 94.5 | 32.0 | 339 |
| Unprocessed-1 | 91.4 | 34.4 | 376 |
| Unprocessed-2 | 90.2 | 37.9 | 420 |
| Unprocessed-3 | 92.1 | 40.0 | 433 |
| Unprocessed-4 | 91.0 | 40.2 | 442 |
| Processed-1 | 133.4 | 37.8 | 284 |
| Processed-2 | 131.5 | 45.0 | 342 |
| Processed-3 | 128.5 | 38.9 | 303 |
| Processed-4 | 131.1 | 39.4 | 301 |

The abbreviations in Table 4 are explained under Table 3.

As described above, HIV cultures were established using 0.5 ml aliquots of unprocessed and processed study samples. P24 antigen levels (pg/ml) from the study samples on day 4 and day 7 of culture are shown in Table 5.

TABLE 5

| | p24 | p24 |
|---|---|---|
| Sample/Number | Day 4 | Day 7 |
| Unprocessed-1 | 1360 | 484 |
| Unprocessed-2 | 1180 | 418 |
| Unprocessed-3 | 1230 | 516 |
| Unprocessed-4 | 1080 | 563 |
| Processed-1 | 579 | 241 |
| Processed-2 | 760 | 303 |
| Processed-3 | 590 | 276 |
| Processed-4 | 622 | 203 |

Finally, one unprocessed sample and one processed sample were selected for the performance of direct viral titration without culture. The results are shown in Table 6.

TABLE 6

| Sample/Number | Titer (log 10 ml) |
|---|---|
| Unprocessed-1 | 1.5 |
| Processed-1 | 0.0 |

The red blood cells were minimally affected by the process, although some reproducible macrocytosis was observed. Although on co-culturing of processed samples, there appeared to be some residual live virus, this was not confirmed by direct titration experiments.

### Example 6

The objective of this experiment was to evaluate the toxicity of the proces towards red blood cells in a comprehensive manner.

### Methods

For this experiment, 1 ml of anticoagulated blood was obtained from the same HIV-seronegative donor as in the first experiment. The blood was centrifuged and the plasma was removed. The remaining cell pellet was resuspended in 10 ml of the CPD buffer and centrifuged. This washing process was repeated a total of three times. The final pellet was resuspended in 20 ml of the SCPD buffer and distributed into plastic tubes in 2 ml aliquots with all 10 aliquots being retained for further manipulation. Eight tubes were subjected to the process, while the final two tubes were retained as control, unprocessed tubes. After the processing, all the tubes were centrifuged, and the resulting pellet was resuspended in 100 μl buffer. A complete hemogram was performed on these reconcentrated study samples.

As in the first experiment, a separate aliquot of blood was obtained from the donor when the study sample was taken. A complete hemogram was performed on this baseline sample. As the study samples were re-concentrated to 33-50% of their original state, more direct comparisons with the baseline sample could be undertaken than were possible in our earlier experiment.

### Results

Red blood cell parameters for the baseline sample as well as or the unprocessed and processed study samples are shown in Table 7. The abbreviations used in Table 7 are defined in Table 3.

TABLE 7

| Sample/Number | RBC | HGS | MCV | MCH | MCHC |
|---|---|---|---|---|---|
| Baseline | 4.76 | 152 | 94.9 | 31.9 | 336 |
| Unprocessed-1 | 0.99 | 33 | 90.2 | 33.0 | 366 |
| Unprocessed-2 | 1.08 | 41 | 89.5 | 38.3 | 427 |
| Processed-1 | 1.15 | 34 | 153.0 | 29.9 | 195 |
| Processed-2 | 1.15 | 34 | 155.9 | 29.4 | 189 |
| Processed-3 | 1.26 | 28 | 161.5 | 22.1 | 137 |
| Processed-4 | 0.79 | 24 | 158.4 | 30.8 | 194 |
| Processed-5 | 0.54 | 29 | 162.5 | 54.5 | 335 |
| Processed-6 | 1.04 | 32 | 163.0 | 31.3 | 192 |
| Processed-7 | 1.35 | 45 | 144.7 | 33.0 | 228 |
| Processed-8 | 1.22 | 45 | 135.8 | 36.5 | 269 |

There was macrocytosis of the cells which was present in all the processed samples. Comparable hemoglobin levels were measured in the unprocessed and processed samples. The absolute values were appropriate for the residual dilution. The red blood cells are preserved.

### Example 7

### Methods

For this experiment, 5 ml of anticoagulated blood was obtained from the same HIV-seronegative donor as in the first two experiments. The blood was centrifuged, and the plasma was removed. The remaining cell pellet was resuspended in 100 ml of the CPD buffer, and centrifuged. This washing process was repeated a total of three times. The final pellet was resuspended in 100 ml of the SCPD buffer and distributed in 25 ml aliquots, in T25 tissue culture flasks, with all four aliquots being retained for further manipulation. Two flakes were subject to the process, while the other two were retained as control, unprocessed flasks. After the processing, the contents of each of the flasks was observed and a visual determination of the cells' capacity to absorb oxygen (turning a brighter red on exposure to ambient air) was made. Following this, the contents of the flasks were aspirated and centrifuged, with the residual pallet resuspended in a small volume of buffer. A complete hemogram was performed on these re-concentrated study samples.

As in Examples 5 and 6, a separate aliquot of blood was obtained from the donor when the study sample was taken. A complete hemogram was performed on this baseline sample. As the study samples were re-concentrated to 33-50% of their original state, direct caparisons [should this be “comparisons”?] of a number of specific parameters would be possible with the baseline sample.

### Results

On visual inspection, there were no appreciable differences between the processed and unprocessed study samples. Specifically, there appeared to be a uniform distribution of well suspended cells. On exposure to ambient air, the contents of all flasks became somewhat brighter red. No specific quantitative measurements of oxygenation were made.

Red blood cell parameters for the baseline sample as well as for the unprocessed and processed study samples are shown in Table 8. The abbreviations used in Table 8 are defined under Table 3.

TABLE 8

| Sample/Number | RBC | HGS | MCV | MCH | MCHC |
|---|---|---|---|---|---|
| Baseline | 4.75 | 153 | 95.0 | 32.3 | 339 |
| Unprocessed-1 | 0.93 | 30 | 151.5 | 32.3 | 213 |
| Unprocessed-2 | 0.92 | 30 | 155.5 | 32.1 | 207 |
| Processed-1 | 0.82 | 27 | 156.5 | 32.8 | 209 |
| Processed-2 | 0.81 | 26 | 152.6 | 32.4 | 212 |

This experiment was designed to more closely approximate conditions of red blood cells to be transfused into a patient, and was consequently conducted at higher volumes. On a preliminary basis, it does not appear that the process impairs the red blood cells' ability to carry oxygen, although this should be measured more formally. Interestingly, in this experiment, there was no difference in cell size between the processed and unprocessed samples, both being large compared to baseline. Comparable hemoglobin levels were measured in all the study samples.

### Example 8

In this experiment, Immunoglobulin G (IgG) was irradiated in lyophilized form.

### Method

The results of SPLC analysis of IgG are given in Table 9. AS the results demonstrate, the product appears to be unaffected after being irradiated to a dose of 25 kGy at room temperature when the irradiation is delivered at a rate of approximately 0.7 kGy/hr. This has not been previously demonstrated.

TABLE 9

| Parameter | Control (%) | Irradiated (%) |
|---|---|---|
| Polymer (must be >2%) | 1 | 1 |
| Dimer | 10 | 13 |
| Monomer | 88 | 84 |
| Low Molecular Weight | 1 | 2 |

The results presented by Gergely, et al., using freeze dried IgG showed that a portion of the protein was insoluble after an irradiation dose of 12 kGy to 25 kGy at standard irradiation dose rates. (Gergely, J., et al., “Studies of Gama-Ray-Irradiated Human Immunoglobulin G.” SM-92/12 I.A.E.A.) In contrast, using the present method at a dose rate of approximately 0.7 kGy/hr, none of the protein was insoluble. This would indicate that little or no change or degradation of the protein occurred. Further, Gergely, et al., found that a liquid formulation of human IgG lost all of its activity after irradiation. In studies using the present method on intravenous immunoglobulin (IVIG) in liquid form, it was shown that greater than 70% of a specific antibody in hyperimmune IVIG was retained.

### Example 9

In this experiment, alpha I proteinase inhibitor and fibrinogen were irradiated in lyophilized form.

### Method

The samples were placed in a Gamma cell 220 and irradiated according to the present process to a total dose of 25 kGy. Samples were then returned to the laboratory for analysis. The dose rate was 0.72 kGy/hr.

### Results

The alpha 1 proteinase inhibitor, both treated and control, were 40% of a standard normal pooled plasma sample. The Mancini radial immunodifusion technique was used as the assay.

The topical fibrinogen complex vials were reconstituted in 10 ml of water. Protamine sulphate vials were reconstituted in 10 ml of water. Protamine sulphate at a concentration of 10 mg/ml was added to the samples. There was instant formation of monomer in all three preparations.

### Example 10

In this experiment, Factors VII, VIII and IV were irradiated in lyophilized form.

### Method

The samples were placed in a Gamacell 220 and irradiated to various total doses at a dose rate of approximately 1 kGy/hr.

### Results

Factor VII retained 67% activity at 20 kGy and 75% at 10 kGy. Facror VIII retained 77% activity at 20 kGy and 88% at 10 kGy. Similarly, Factor IV showed an activity level of 70% at 20 kGy and 80% at 10 kGy.

### Analysis

Excellent results were found for the three Factors. To our knowledge, no one has been able to achieve these results by irradiating the Factors at ambient temperature to such a high dose of radiation with such little loss of activity. This is in direct contrast with the results of Kitchen, et al., “Effect of Gamma Irradiation on the Human Immunodeficiency Virus and Human Coagulation proteins,” Vox Sang 56:223-229 (1989), who found that “the irradiation of lyophilized concentrates is not a viable procedure.” Similarly, Hiemstra, et al., “Inactivation of human immunodeficiency virus by gamma radiation and its effect on plasma and coagulation factors,” Transfusion 31:32-39 (1991), also concluded that “Gamma radiation must be disregarded as a method for the sterilization of plasma and plasma-derived products, because of the low reduction of virus infectivity at radiation doses that still give acceptable recovery of biologic activity of plasma components.”

### Example 11

In this experiment, red blood cells were irradiated at a dose rate of 0.5 kGy/hr for periods of time ranging from 7.5 to 90 minutes in order to remove bacterial contaminants.

### Method

Red blood cells were collected from a healthy donor in EDTA, washed 3 times with CPD solution and resuspended in DPC to provide a 1:20 dilution based on the original blood volume. The cell suspension was then subdivided into 14 tubes. To seven of the tubes, approximately 1.0×10⁴ Staphylococcus epidermidia were added. The cells were placed on ice for transport to the irradiation facility. All of the samples were placed in the chamber at ambient temperature and irradiated at 0.5 kGy/hr for periods of time to give total doses of 0.625, 0.125, 0.250, 0.375, 0.500 and 0.750 kGy, respectively. The samples were removed and gitated at each time point and placed on ice for transport either to the microbiology lab or the hematology lab for analysis.

### Results

The results of the microbiology assays are given in Table 10.

TABLE 10

| Radiation Dose (kGy) | Time (Min.) | Number Surviving |
|---|---|---|
| 0 | | 92,200 |
| 0.625 | 7.5 | 84,500 |
| 0.125 | 15 | 35,000 |
| 0.250 | 30 | 10,067 |
| 0.375 | 45 | 1,800 |
| 0.500 | 60 | 250 |
| 0.750 | 90 | 0 |

Thus, a dose of 0.75 kGy provides a 4.5 log₁₀ reduction in bacterial survivors. This represents a significant safety Factor For blood. Further, the D10 value is approximately 0.125 kGy which corresponds well with the values reported in the literature for similar species of staphylococcus (B. A. Bridges, “The effect of N-Ethylmaleimide on the radiation sensitivity of bacteria,” J. Gen. Microbiol. 26:467-472 (1962), and Jacobs, G. P. and Sadeh, N., “Radiosensitization of Staphylococcus aureus by p-hydroxybenzoic acid,” Int. J. Radiat. Biol. 41:351-35.6 (1982).

In order to demonstrate that the red blood cells remained viable after the irradiation process, the following parameters were determined for the cells, WBC, Neutrophils, Lymphocytes, Monocytes, Eosinophils and Basophils. These determinations merely enumerated the number of cells present. All nucleated cells would, of course, be inactivated by the radiation dose delivered. The other red blood cell parameters monitored are listed in Table 11. The Methaemoglobin value was unchanged from that of the controls even after a radiation dose of 0.75 kGy. This experiment demonstrates that red blood cells can be safely irradiated by the present method to a dose of 0.75 kGy at room temperature with no loss of cell function.

### Example 12

This experiment was conducted using the method in Example 11 to confirm the findings of Example 11 and to expand upon some of the parameters measured. The results of this experiment are given in Table 12.

### Results

### (See Table 12, Below.)

These results confirm the previous results and indicate that indeed, red blood cells can be irradiated to a dose sufficient to provide 4.5 log₁₀ reduction in bacterial count.

It is contemplated that future experiments will provide similar results for platelet. Thus, with little or no additional manipulation, and without the addition of extraneous materials, red blood cells can be treated by the present process to provide a bacteriologically safe product, thus further reducing the risk of untoward reactions in recipients.

TABLE 11
Red Blood Cell Valus as a Function of Radiation Dose Received

| | Total Dose (in kGy) | | | | | |
|---|---|---|---|---|---|---|
| | Whole | | | | | |
| Parameter | Blood | 0 | 0.625 | 0.125 | 0.250 | 0.500 |
| RBC | 5.06 | 1.49 | 1.27 | 1.77 | 1.73 | 1.43 |
| HGB | 153 | 43 | 41 | 56 | 56 | 46 |
| HTC | .483 | .142 | .120 | .156 | .163 | 1.31 |
| MCV | 95.5 | 95.6 | 94.3 | 94.2 | 93.7 | 32.1 |
| MCH | 31.2 | 31.1 | 32.2 | 31.7 | 32.2 | 32.5 |
| MCHC | 327 | 325 | 341 | 336 | 344 | 353 |
| RDW | 13.93 | 12.1 | 12.7 | 12.9 | 12.9 | 13.2 |
| METHgB | 0.9 | 0.3 | 0.3 | 0.3 | 0.0 | 0.9 |

TABLE 12
Red Blood Cell Values as a Function of Radiation Dose Received

| | Total Dose (in kGy) | | | | | | |
|---|---|---|---|---|---|---|---|
| Parameter | 0 | 0.625 | 0.125 | 0.250 | 0.375 | 0.555 | 0.750 |
| HGB | 1.8 | 1.7 | 1.8 | 1.7 | 2.0 | 2.0 | 2.0 |
| % O | 96.6 | 96.5 | 96.2 | 96.3 | 96.4 | 96.5 | 96.0 |
| % CO | 1.0 | 1.2 | 1.6 | 1.3 | 1.7 | 1.5 | 1.5 |
| % NET | 0.5 | 0.5 | −0.5 | 0.4 | −0.2 | 0.4 | 0.8 |
| % Reduced | 1.9 | 1.9 | 2.7 | 2.4 | 3.2 | 1.7 | 1.7 |
| p60 (mm Hg) | 34 | nd | nd | nd | nd | nd | 24 |
| [symbol] | 2.1 | nd | nd | nd | nd | nd | 1.8 |
| Coefficient | | | | | | | |

nd = not done
The uncertainty with the methaemoglobin levels is ±2%; with the p50 it is ±4% (95% confidence).

Having now fully described this invention, it will be understood to those of ordinary skill in the art that the methods of the present invention can be carried out with a wide and equivalent range of conditions, formulations, and other parameters without departing from the scope of the invention or any embodiments thereof. All patents and publications cited herein are hereby fully incorporated by reference in their entirety.

What is claimed is:

1. A method for sterilizing a preparation containing a blood component that is sensitive to ionizing radiation, said method comprising:
(i) reducing the residual solvent content of a preparation containing a blood component to a level effective to protect said preparation containing a blood component from said ionizing radiation; and
(ii) irradiating said preparation containing a blood component with a suitable ionizing radiation at an effective rate for a time effective to sterilize said preparation containing a blood component, wherein said effective rate is not constant for the duration of the sterilization procedure.

2. A method for sterilizing a preparation containing a blood component that is sensitive to ionizing radiation, said method comprising:
(i) adding to a preparation containing a blood component at least one stabilizer in an amount effective to protect said preparation containing a blood component from said ionizing radiation; and
(ii) irradiating said preparation containing a blood component with a suitable ionizing radiation at an effective rate for a time effective to sterilize said preparation containing a blood component, wherein said effective rate is not constant for the duration of the sterilization procedure.

3. A method for sterilizing a preparation containing a blood component that is sensitive to ionizing radiation, said method comprising:
(i) reducing the residual solvent content of a preparation containing a blood component to a level effective to protect said preparation containing a blood component from said ionizing radiation;
(ii) adding to said preparation containing a blood component at least one stabilizer in an amount effective to protect said preparation containing a blood component from said ionizing radiation; and
(iii) irradiating said preparation containing a blood component with a suitable ionizing radiation at an effective rate for a time effective to sterilize said preparation containing a blood component, wherein (i) and (ii) may be performed in any order and said effective rate is not constant for the duration of the sterilization procedure.

4. The method according to claim 1 or 3, wherein said solvent is water.

5. The method according to claim 1 or 3, wherein said solvent is an organic solvent.

6. The method according to claim 1, 2 or 3, wherein said ionizing radiation is gamma radiation.

7. The method according to claim 1, 2 or 3, wherein said effective rate comprises a rate of not more than 3.0 kGy/hour.

8. The method according to claim 1, 2 or 3, wherein said effective rate comprises a rate of more than 3.0 kGy/hour.

9. The method according to claim 1, 2 or 3, wherein said effective rate comprises a rate of not more than 6.0 kGy/hour.

10. The method according to claim 1, 2 or 3, wherein said effective rate comprises a rate of not more than 18.0 kGy/hour.

11. The method according to claim 1, 2 or 3, wherein said effective rate comprises a rate of not more than 30.0 kGy/hour.

12. The method according to claim 1, 2 or 3, wherein said preparation containing a blood component is maintained in a low oxygen atmosphere.

13. The method according to claim 12, wherein said preparation containing a blood component is maintained in an argon atmosphere.

14. The method according to claim 1 or 3, wherein said residual solvent content is reduced by lyophilization.

15. The method according to claim 1 or 3, wherein said residual solvent content is less than 2.0%.

16. The method according to claim 1 or 3, wherein said residual solvent content is less than 1.0%.

17. The method according to claim 1 or 3, wherein said residual solvent content is less than 0.5%.

18. The method according to claim 2 or 3, wherein said at least one stabilizer comprises at least one antioxidant.

19. The method according to claim 2 or 3, wherein said at least one stabilizer comprises at least one free radical scavenger.

20. The method according to claim 2 or 3, wherein said at least one stabilizer comprises a member selected from the group consisting of ascorbic acid, or a salt or ester thereof; DMSO; trehalose; mannitol, glutathione; tocopherol; polyhydric alcohols; flavonoids; and combinations of two or more thereof

21. The method according to claim 1, 2 or 3, wherein said effective rate comprises a rate of about 3.0 kGy/hr.

22. The method according to claim 21, wherein said effective rate further comprises a rate of about 2.0 kGy/hr.

23. The method according to claim 1, 2 or 3, wherein said preparation containing a blood component is irradiated at ambient temperature.

24. The method according to claim 1, 2 or 3, wherein said preparation containing a blood component is irradiated at a temperature below ambient temperature.

25. The method according to claim 1, 2 or 3, wherein said preparation containing a blood component is irradiated at a temperature below the eutectic point of said preparation containing a blood component.

26. The method according to claim 1, 2 or 3, wherein said preparation containing a blood component comprises a member selected from the group consisting of cellular blood components, blood proteins, liquid blood components and combinations of two or more thereof.

27. The method according to claim 1, 2 or 3, wherein said preparation containing a blood component comprises a cellular blood component.

28. The method according to claim 27, wherein said cellular blood component is selected from the group consisting of red blood cells, white blood cells, platelets and combinations of two or more thereof.

29. The method according to claim 1, 2 or 3, wherein said preparation containing a blood component comprises a blood protein.

30. The method according to claim 29, wherein said blood protein is selected from the group consisting of blood clotting factors, enzymes, plasminogen, fibrinogen, immunoglobulins and combinations of two or more thereof.

31. The method according to claim 29, wherein said blood protein is selected from the group consisting of Factor I, Factor II, Factor III, Factor IV, Factor V, Factor VI, Factor VII, Factor VIII, Factor IX, Factor X, Factor XI, Factor XII, Factor XIII, von Willebrands Factor, Factor Ia, Factor IIa, Factor Va, Factor VIa, Factor VIIa, Factor VIIIa, Factor IXa, Factor Xa, Factor XIIIa and combinations of two or more thereof.

32. The method according to claim 1, 2 or 3, wherein said preparation containing a blood component comprises a liquid blood component.

33. The method according to claim 32, wherein said liquid blood component is selected from the group consisting of plasma and serum.

34. The method according to claim 32, wherein said liquid blood component is serum.

35. The method according to claim 32, wherein said liquid blood component is plasma.

36. The method according to claim 1, 2 or 3, wherein at least one sensitizer is added to said preparation containing a blood component prior to irradiating.
